(19) United States
(12) Patent Application Publication
(10) Pub. No.: US 2016/0274123 A1
(43) Pub. Date: Sep. 22, 2016

(54) One-Photon and/or Two-Photon Fluorescent Probe for Sensing Hydrogen Sulfide, Imaging Method of Hydrogen Sulfide Using Same, and Manufacturing Method Thereof

(71) Applicant: POSTECH ACADEMY-INDUSTRY FOUNDATION, Gyeongsangbuk-do (KR)

(72) Inventors: Kyo Han Ahn, Gyeongsangbuk-do (KR); Dokyoung Kim, Busan (KR); Subhankar Singha, Gyeongsangbuk-do (KR)

(21) Appl. No.: 15/037,168

(22) PCT Filed: Feb. 26, 2014
(86) PCT No.: PCT/KR2014/001589

§ 371 Date: May 17, 2016

(30) Foreign Application Priority Data
Nov. 18, 2013 (KR) 10-2013-0140017

(51) Int. Cl. G01N 33/58

(52) U.S. Cl. CPC G01N 33/582

(57) ABSTRACT

The present invention relates to a one-photon and/or two-photon fluorescent probe for selectively detecting hydrogen sulfide in the human body using a compound including an α,β-unsaturated carbonyl group and an acedan (2-acyl-6-dimethyl-amino-naphthalene) fluorescent material; to an imaging method of hydrogen sulfide in cells using the same; and to a manufacturing method of the fluorescent probe. More specifically, in the fluorescent probe of the present invention, the α,β-unsaturated carbonyl group of the compound selectively binds to hydrogen sulfide, inducing an increase in fluorescence of the acedan fluorescent material. The fluorescent probe according to the present invention can be conveniently synthesized, enables two-photon excitation, and corresponds to a small-molecule probe having stability and low toxicity in the body. In addition, the fluorescent probe according to the present invention can exhibit a fluorescent change by selectively reacting with hydrogen sulfide, thereby imaging the distribution of hydrogen sulfide in cells or tissues, and thus can be useful for a composition for imaging and an imaging method.

### CROSS-REFERENCE TO RELATED APPLICATION

This application claims priority to and the benefit of Korean Patent Application No. 10-2013-0140017, filed on Nov. 18, 2013 and International Patent Application No. PCT/KR2014/001589, filed on Feb. 26, 2014, the disclosure of which is incorporated herein by reference in its entirety.

The present invention was undertaken with the support of Global Research Laboratory Program No. NRF-2014K1A1A2064569 grant funded by the National Research Foundation of Korea(NRF) funded by the Ministry of Science, and ICT & Future Planning and Korea Health Technology R&D Project No. HI13C1378 grant funded by the Ministry of Health & Welfare, Republic of Korea.

### TECHNICAL FIELD

The present invention relates to a probe for selectively sensing hydrogen sulfide in the living body using a compound having an α,β-unsaturated carbonyl group and a 2-acyl-6-dimethyl-amino-naphthalene (acedan) fluorescent substance, and a method of manufacturing the probe.

### BACKGROUND ART

Hydrogen sulfide (H₂S) is a substance in equilibrium with an anion (HS⁻) thereof under physiological conditions, and a gas compound significantly involved in signal transduction in addition to carbon monoxide and nitrogen oxide. It has been reported that hydrogen sulfide is associated with various physiological procedures to modulate neuronal activity, to relax smooth muscle, to regulate an insulin release, to induce angiogenesis, to suppress inflammation, etc. To confirm biological phenomena shown by such hydrogen sulfide and identify the characteristics thereof, various analysis methods have been suggested. As an example, the “methylene blue” method is used for analyzing hydrogen sulfide through the change in absorption in the presence of an iron oxidant, and the “auto-analysis method for a silver/sulfide ion electrode film” is an electrochemical analysis method by potential difference. However, such analysis methods are not suitable for an in vivo analysis for sensing hydrogen sulfide in the living body, and need sample preparation and pre-treatment even for an in vitro analysis. Accordingly, for the in vivo analysis, there is a demand for the development of a fluorescent probe enabling noninvasive detection with high sensitivity.

Recently, various fluorescent probes using high nucleophilicity, which is the unique characteristic of hydrogen sulfide, are being developed. Things to be considered in priority in the development of such fluorescent probes are as follows: (1) high selectivity, which is not subjected to interference from a sulfide having a high concentration in the living body, for example, glutathione (GHS), cysteine (Cys) or homocysteine (Hcy), (2) high sensitivity to sense hydrogen sulfide in cells, (3) a high response rate, (4) low cytotoxicity, and (5) an ability of imaging a biological tissue.

Meanwhile, all of the systems for a hydrogen sulfide-sensing fluorescent probe, which have been reported so far, realize a fluorescent change using chemical reactions (substitution and reduction). (1) Arylazide (ArN₃) compounds are converted into arylamine (aryl-NH₂) by hydrogen sulfide, resulting in a fluorescence turn-on phenomenon. While various fluorescent probes have been reported (Yu, F.; Li, P.; Song, P.; Wang, B.; Zhaoa, J.; Han, K. Chem. Commun. 2012, 48, 2852./Montoya, L. A.; Pluth, M. D. Chem. Commun. 2012, 48, 4767), a fluorescence sensing method for hydrogen sulfide using arylazide has low selectivity in response to competitive biothiol as well as a low response rate. (2) Arylsulfonyl azide quickly responds to hydrogen sulfide due to a higher electrophilicity than arylazide, but exhibits very low substrate selectivity. Particularly, the interference of glutathione, which is the most biologically abundant sulfide, causes a serious problem during the development of a hydrogen sulfide-selective fluorescent probe.

To overcome such problems, recently, a system based on disulfide exchange, and sensing systems based on 1,4-addition, which is conjugate addition followed by an intramolecular ester hydrolysis reaction, are reported. However, these systems cannot sense hydrogen sulfide in the living body due to low sensitivity.

### DISCLOSURE

### Technical Problem

Therefore, to overcome problems of the conventional art, the inventors developed a molecular probe enabling fluorescence imaging for hydrogen sulfide in the living body, thereby completing the present invention.

Accordingly, the objective of the present invention is to provide a novel one-photon and/or two-photon fluorescent probe, a method of manufacturing the probe, and an imaging method for hydrogen sulfide in cells using the probe.

However, the technical subject to be accomplished by the present invention is not limited to the above-described objective, and other subjects not described herein will be clearly understood by those of ordinary skill in the art with reference to the following descriptions.

### Technical Solution

To accomplish the objective of the present invention, the present invention provides a one-photon and/or two-photon fluorescent probe represented by Formula 1.

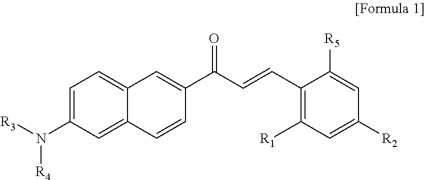

Here, in Formula 1, R₁ is hydrogen, an alkyl, or a substituted C₁₋₃ alkyl, R₂ is hydrogen, an alkyl, or a substituted C₁₋₃ alkyl, R₃ is hydrogen, an alkyl, or a substituted C₁₋₃ alkyl, R₄ is hydrogen or an alkyl, and R₅ is CHO or COCF₃.

In an exemplary embodiment of the present invention, in Formula 1, R₁ may be hydrogen or methoxy (OCH₃), R₂ may be hydrogen or methoxy (OCH₃), R₃ may be ethanol (CH₂CH₂OH), R₄ may be hydrogen, and R₅ may be CHO.

In another exemplary embodiment of the present invention, the probe may bind to hydrogen sulfide, thereby exhibiting fluorescence.

Also, the present invention provides an imaging method for hydrogen sulfide in cells, which includes injecting the one-photon and/or two-photon fluorescent probe into a cell, reacting the injected fluorescent probe with hydrogen sulfide in the cell, thereby exhibiting fluorescence, and observing the fluorescence using a one-photon or two-photon fluorescence microscope.

In addition, the present invention provides a method of manufacturing a one-photon and/or two-photon fluorescent probe for detecting hydrogen sulfide by introducing a methoxy group to R₁ and/or R₂ of Formula 1.

### ADVANTAGEOUS EFFECTS

A fluorescent probe of the present invention has a two-photon excitable property which is excited to an excited state using energy corresponding to the half of a one-photon excitation. Therefore, the fluorescent probe has advantages of deeper tissue penetration and low cell destruction, and is less affected by quenching of hemoglobin in the living body, and only the focal area thereof is excited, resulting in very high-resolution images.

### DESCRIPTION OF DRAWINGS

FIG. 1 shows a fluorescence change when Compound 2 according to the present invention reacts with hydrogen sulfide at various concentrations.

FIG. 2 shows a fluorescence change over time when Compound 2 according to the present invention reacts with hydrogen sulfide.

FIG. 3 shows fluorescence changes when Compound 2 according to the present invention reacts with hydrogen sulfide and biological sulfides (cysteine, homocysteine and glutathione).

FIG. 4 shows fluorescence changes when Compound 2 according to the present invention reacts with various types of biological substances.

FIG. 5 shows the sensitivity of Compound 2 according to the present invention with respect to hydrogen sulfide, which is assessed by the fluorescence change.

FIG. 6 shows the effect of acidity (pH) when Compound 2 according to the present invention reacts with hydrogen sulfide.

FIG. 7 shows results of a cell imaging experiment for Compound 2 (Cpd 2) according to the present invention using one-photon and two-photon fluorescence microscopes.

FIG. 8 shows results of a mouse organ tissue imaging experiment for Compound 2 according to the present invention using a two-photon fluorescence microscope.

FIG. 9 shows results of a fish organ tissue imaging experiment for Compound 2 according to the present invention using a two-photon fluorescence microscope.

FIG. 10 shows the cytotoxicity of Compound 2 according to the present invention.

FIG. 11 shows results of quantum chemical calculation to assess the hydrogen sulfide selectivity of Compounds 2, 3 and 4 according to the present invention.

FIG. 12 shows the hydrogen sulfide selectivity of Compounds 2, 3 and 4 according to the present invention.

### MODES OF THE INVENTION

The present invention is directed to providing a one-photon and/or two-photon fluorescent probe represented by Formula 1.

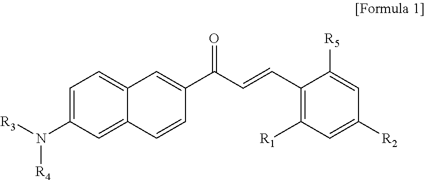

In Formula 1, R₁ may be hydrogen, an alkyl, or a substituted C₁₋₃ alkyl, R₂ may be hydrogen, an alkyl, or a substituted C₁₋₃ alkyl, R₃ may be hydrogen, an alkyl, or a substituted C₁₋₃ alkyl, R₄ may be hydrogen or an alkyl, and R₅ may be CHO or COCF₃, and like Formula 18, most preferably, R₁ is hydrogen or methoxy (OCH₃), R₂ is hydrogen or methoxy (OCH₃), R₃ is ethanol (CH₂CH₂OH), R₄ is hydrogen, and R₅ is CHO, but the present invention is not limited thereto.

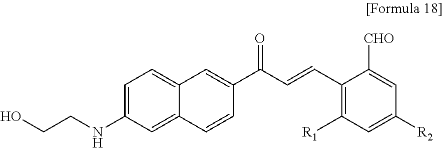

The term “alkyl” refers to an aliphatic hydrocarbon group. In the present invention, the alkyl is used as the concept including all of the “saturated alkyls” including no alkene or alkyne moiety, and the “unsaturated alkyls” including at least one alkene or alkyne moiety. The alkyl may be, but is not particularly limited to, a substituted C₁₋₃ alkyl.

The present inventors newly developed a fluorescent probe including an α,β-unsaturated carbonyl group, which has an aryl (2-formyl-4,6-dimethoxyphenyl) group having abundant electrons and steric hindrance, and a 2-acyl-6-dimethyl-amino-naphthalene (acedan) fluorescent substance. In the structure of the fluorescent probe compound developed in the present invention, the unsaturated carbonyl group reacts with hydrogen sulfide with high selectivity and sensitivity, the acedan fluorescent substance providing a fluorescent signal is a substance having a two-photon excitable property and an excellent performance in cell and tissue imaging using a two-photon fluorescence microscope.

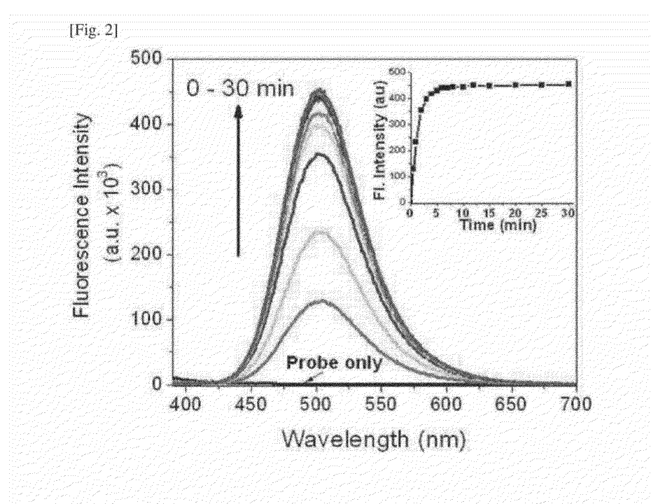

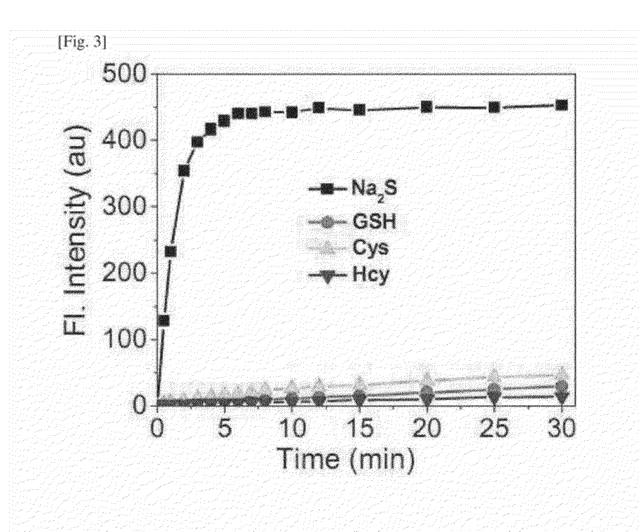

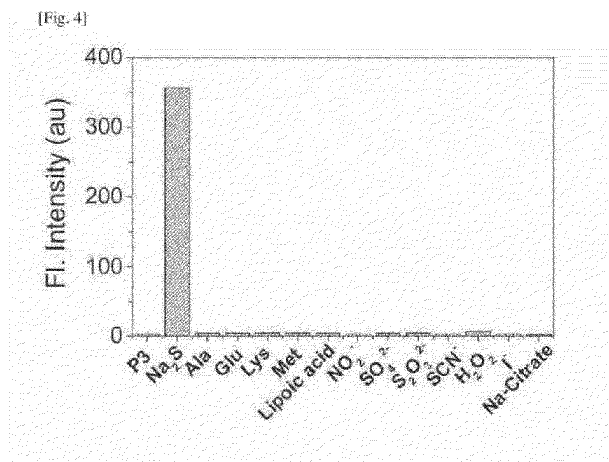

The compound of the probe has a fluorescence change according to 1,4-addition (Michael addition) between the hydrogen sulfide and the α,β-unsaturated carbonyl group, and selectively binds to the hydrogen sulfide among various sulfide substances in the living body, thereby exhibiting fluorescence. That is, the α,β-unsaturated carbonyl group of the probe according to the present invention reacts with hydrogen sulfide by 1,4-addition, thereby inducing a fluorescence turn-on phenomenon of the acedan fluorescent substance, and thus only hydrogen sulfide is detected with high selectivity and sensitivity among various types of sulfides and biological substances. In an exemplary embodiment of the present invention, as a result of observing fluorescence changes over time by adding various sulfides (hydrogen sulfide, cysteine, homocysteine and glutathione) to a buffer along with the probe of the present invention, it is confirmed that the probe of the present invention selectively reacts with hydrogen sulfide (refer to FIGS. 2 and 3). Also, as a result of observing selectivity under biological conditions (amino acids, reactive oxygen, etc.) excluding a sulfide, it is confirmed that the fluorescence turn-on phenomenon is selectively observed only in hydrogen sulfide (refer to FIG. 4).

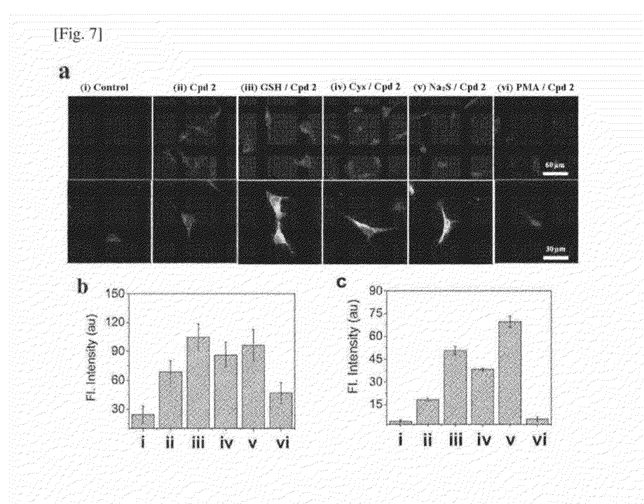

Among the cell and tissue imaging methods, two-photon fluorescence microscopy, compared to one-photon fluorescence microscopy, is advantageous in terms of deeper tissue penetration, lower cell destruction, and quenching caused by a lower hemoglobin level in the living body. In one exemplary embodiment of the present invention, as a result of imaging the distribution of hydrogen sulfide in cells and tissues using the probe of the present invention using the two-photon fluorescence microscopy, it is confirmed that hydrogen sulfide in cells and tissues is imaged with excellent efficiency using the probe of the present invention (refer to FIGS. 7, 8 and 10).

Therefore, the present invention may provide a method of imaging hydrogen sulfide in cells, which includes: (a) injecting the fluorescent probe into cells; (b) reacting the injected fluorescent probe with hydrogen sulfide in biological cells to show fluorescence; and (c) observing the fluorescence using a one-photon or two-photon fluorescence microscope.

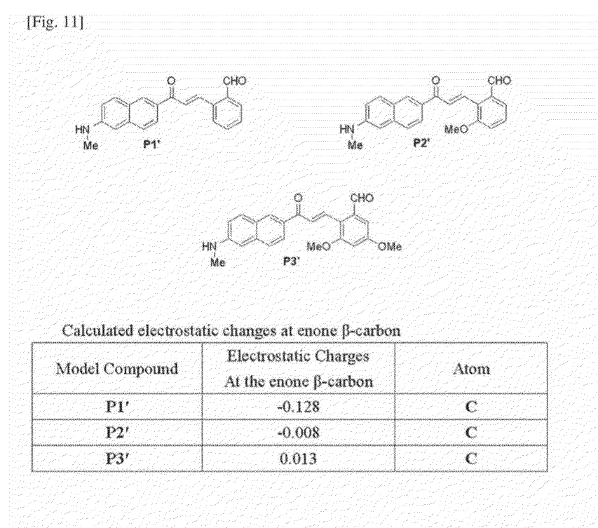

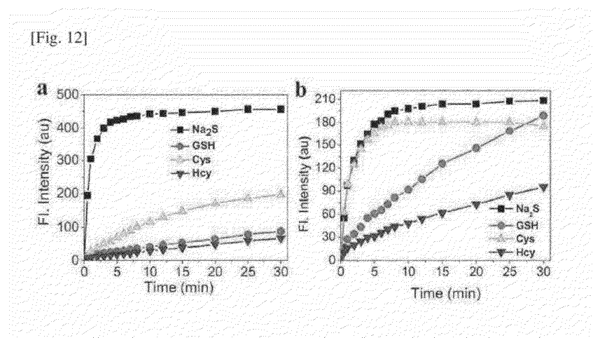

In addition, in one exemplary embodiment of the present invention, as a result of quantum chemical calculation to examine whether or not electron donor groups, most preferably, methoxy groups are needed at ortho and para positions such that an α,β-unsaturated carbonyl group has selectivity to hydrogen sulfide, it is confirmed that an electron density around carbon at a beta position forming an intramolecular hydrogen bond decreased (here, a negative value refers to an increased electron density), and it is seen that, due to such an effect of the electron density, only the hydrogen sulfide having the highest activity among sulfides can participate in a chemical reaction (refer to FIG. 11). Also, in another exemplary embodiment of the present invention, to confirm the effect of electron donor groups at ortho and para positions, which are methoxy groups, compounds (Formulas 2, 3, and 4) are prepared by substituting one or all of R₁ and R₂ of Formula 1 with a methoxy group or not, and hydrogen sulfide selectivity is checked. As a result, it can be seen that the electron donor group has an effect on the hydrogen sulfide selectivity (refer to FIG. 12).

Accordingly, the present invention provides, as shown in Reaction Formula 1 below, a method of manufacturing a one-photon and/or two-photon fluorescent probe for detecting hydrogen sulfide, which includes:

1) preparing a compound of Formula 6 by performing a Heck reaction on a compound of Formula 5 in the presence of a palladium catalyst, and performing a

Bucherer reaction with 2-aminoethanol;

2) preparing a compound of Formula 8 by performing esterification on a compound of Formula 7 in the presence of an acid catalyst, and sequentially performing bromination and a redox reaction;

3) preparing a compound of Formula 9 by sequentially performing acetal protection and lithium-formylation on the compound of Formula 8 prepared in step 2);

4) preparing a compound of Formula 10 by performing aldol condensation between the compound of Formula 6 prepared in step 1) and the compound of Formula 9 prepared in step 3); and

5) preparing a compound of Formula 2 by substituting all of R₁ and R₂ of Formula 1 with a methoxy group in a reaction of the compound of Formula 10 prepared in step 4) under an acidic condition.

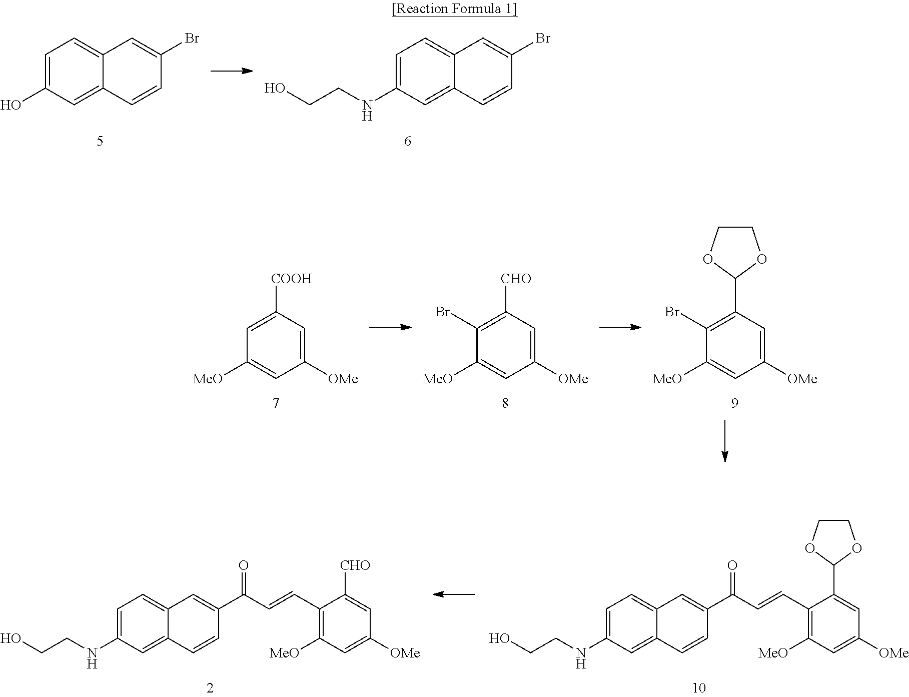

Also, the present invention provides, as shown in Reaction Formula 2 below, a method of manufacturing a one-photon and/or two-photon fluorescent probe for sensing hydrogen sulfide, which includes:

1′) preparing a compound of Formula 12 by performing acetal protection on the compound of Formula 11;

2′) preparing a compound of Formula 13 by performing lithium-formylation on the compound of Formula 12 prepared in step 1′);

3′) preparing a compound of Formula 14 by performing aldol condensation between the compound of Formula 13 prepared in step 2′) and the compound of Formula 6 prepared in step 1); and

4′) preparing a compound of Formula 3 prepared by substituting R₁ of Formula 1 with a methoxy group by a reaction of the compound of Formula 14 prepared in step 3′) under an acidic condition.

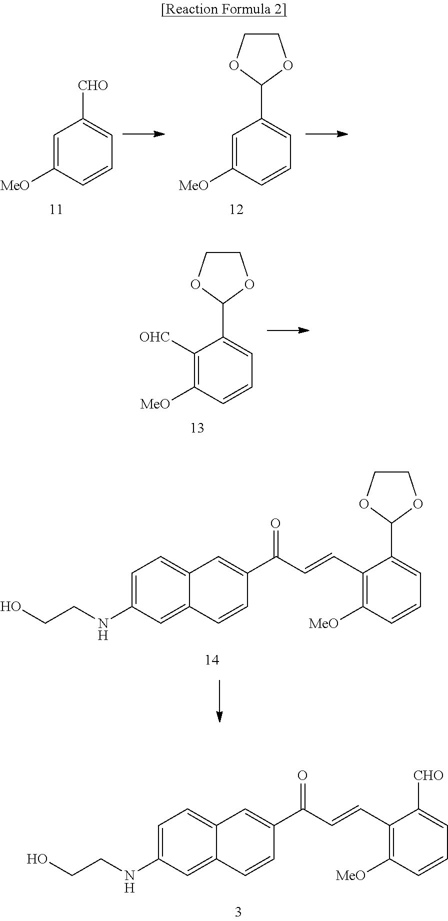 [SMILES: COc1cccc(C=O)c1 ; COc1cccc(C2OCCO2)c1 ; COc1cccc(C2OCCO2)c1C=O ; COc1cccc(C2OCCO2)c1/C=C/C(=O)c1ccc2cc(NCCO)ccc2c1 ; COc1cccc(C=O)c1/C=C/C(=O)c1ccc2cc(NCCO)ccc2c1]

Also, the present invention provides, as shown in Reaction Formula 3 below, a method of manufacturing a one-photon and/or two-photon fluorescent probe for sensing hydrogen sulfide, which includes:

1″) preparing a compound of Formula 16 by sequentially performing acetal protection and lithium-formylation on a compound of Formula 15;

2″) preparing a compound of Formula 17 by performing aldol condensation between the compound of Formula 16 prepared in step 1″) and the compound of Formula 6 prepared in step 1); and

3″) preparing a compound of Formula 4 by substituting R₁ and R₂ of Formula 1 with hydrogen in a reaction of the compound of Formula 17 prepared in step 2″) under an acidic condition.

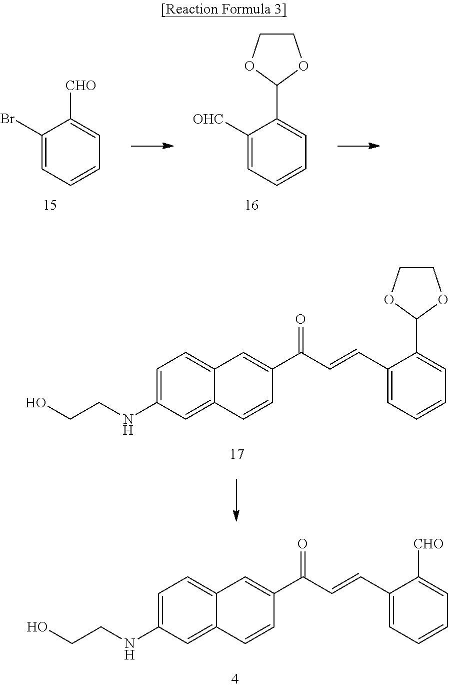 [SMILES: O=Cc1ccccc1Br ; O=Cc1ccccc1C1OCCO1 ; O=C(/C=C/c1ccccc1C1OCCO1)c1ccc2cc(NCCO)ccc2c1 ; O=Cc1ccccc1/C=C/C(=O)c1ccc2cc(NCCO)ccc2c1]

In the present invention, the organic chemical reaction may be performed to prepare the same compound by suitably selecting a reaction solvent, a ligand, a catalyst and/or an additive by those of ordinary skill in the art according to a method known in the art.

Further, the probe according to the present invention may be effectively used to develop a hydrogen sulfide inhibitor by utilizing it to observe a level of hydrogen sulfide through a fluorescent change after cells are treated with an inhibitor for inhibiting hydrogen sulfide. Therefore, the present invention may provide a method of detecting a substance for inhibiting the generation of hydrogen sulfide in the living body using the fluorescent probe of the present invention.

Hereinafter, exemplary examples will be provided to help in understanding the present invention. However, the following examples are merely provided to more easily understand the present invention, but the scope of the present invention is not limited to the following examples.

### Synthesis Example 1

### Synthesis and Structural Analysis of Compound 2

Compound 2 of Formula 2 was synthesized according to the pathway represented by Reaction Formula 1 by the inventors.

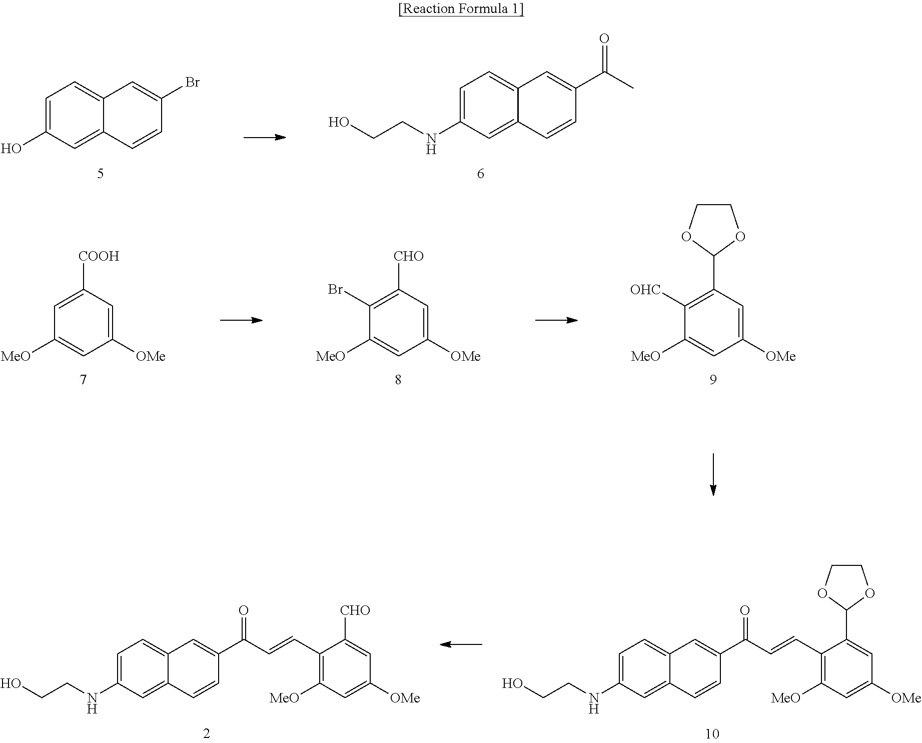

Step 1-1: Synthesis of 1-(6-(2-hydroxyethylamino)naphthalene-2-yl)ethanone

To synthesize Compound 6 of Reaction Formula 1, which is 1-(6-(2-hydroxyethylamino)naphthalene-2-yl)ethanone, first, Compound 5 (6-bromo-2-naphthol, 2 g, 8.97 mmol, Sigma-Aldrich, B73406) as a starting material for synthesis, Pd(OAc)₂ (100 mg, 0.45 mmol) and diphenyl-1-pyrenylphosphine (DPPP, 370 mg, 0.9 mmol) were put into a reaction vessel containing ethylene glycol (15 mL). Subsequently, 2-hydroxylethyl vinyl ether (2.37 g, 27 mmol) and triethylamine (3.12 mL, 22.4 mmol) were put into the reaction vessel, and stirred at 145° C. for 4 hours. After 4 hours, the temperature of a reactant was reduced to room temperature (25° C.), the vessel was open to put dichloromethane (15 mL) and 5% HCl (30 mL) thereinto, and then the resultant mixture was stirred at room temperature for 1 hour. After 1 hour, an organic layer was separated using a separating funnel, dried with Na₂SO₄(5 g), and concentrated using an aspirator (25 ° C., 20˜500 mmHg). In addition, the light yellow solid obtained by the concentration as described above, that is, Compound 5-1, was extracted (developing solvent: 20% EtOAc/Hexane) by column chromatography (diameter: 6 cm, height: 15 cm) using silica gel (Merck-silica gel 60, 230-400 mesh), resulting in a light yellow solid, Compound 5-2 (1.33 g, 80%). 1 H NMR (CDCl₃, 300 MHz, 293 K):δ 8.41 (1 H, s), 7.98 (1 H, dd), 7.87 (1 H, d), 7.70 (1 H, d), 7.16 (1 H, dd), 5.4 (1 H, s), 2.71 (3 H, s).

Afterward, the light yellow solid obtained as described above, Compound 5-2 (1.0 g, 5.37 mmol), 2-aminoethanol (1.64 g, 26.85), Na₂S₂O₅ (2 g, 10.74 mmol), and H₂O (15 mL) were put into a seal-tube, and stirred at 145° C. for 48 hours. After 48 hours, the temperature was reduced to room temperature, the tube was opened, and dichloromethane (200 mL, twice) and H₂O (300 mL) were added to extract an organic layer. The extracted organic layer was dried with Na₂SO₄ (5 g), and concentrated using an aspirator (25° C., 20˜500 mmHg), and then separated (developing solvent: 50:1 v/v dichloromethane-methanol) by column chromatography (diameter: 6 cm, height: 15 cm) using silica gel (Merck-silica gel 60, 230-400 mesh), resulting in a yellow solid, Compound 6 (0.86 g, 70%). 1 H NMR (CDCl₃, 300 MHz, 293 K):δ 8.31 (1 H, s), 7.91 (1 H, dd), 7.72 (1 H, d), 7.60 (1 H, d), 6.94 (1 H, dd), 6.84 (1 H, s), 4.46 (1 H, br.s), 3.94 (2 H, t), 3.44 (2 H, t), 2.67 (3 H, s), 1.66 (1 H, br.$). 13 C NMR (75 MHz, CDCl₃):δ 197.74, 148.56, 138.05, 130.68, 130.63, 130.34, 125.87, 125.82, 124.60, 118.83, 103.45, 60.49, 45.75, 26.39. HRMS-EI (+): m/z calcd for C₁₄H₁₅NO₂: 229.28, found 229.11.

Step 1-2: Synthesis of 2-bromo-3, 5-dimethoxybenzaldehyde

To synthesize Compound 8 of Reaction Formula 1, 2-bromo-3,5-dimethoxy benzaldehyde, first, Compound 7 (5.05 g, 27.7 mmol) as a starting material for synthesis was dissolved in MeOH (100 mL), and a mixture prepared by adding H₂SO₄ (0.2 mL, 3.75 mmol) at 0° C. was refluxed for 20 hours. After 20 hours, the temperature was reduced to room temperature, a saturated NaHCO₃ solution was added to adjust pH to 7, and then residual MeOH was removed using an aspirator (25 ° C., 20˜500 mmHg). In addition, an organic layer was extracted with EtOAc (200 mL, four times), and dehydrated with Na₂SO₄ (10 g) to remove residual water therein. The dried ethylacetate organic layer was concentrated using an aspirator, thereby obtaining Compound 7-1 (5.35 g, 98%), and the following procedure was performed without a separate separating procedure. 1 H NMR (CDCl₃, 300 MHz, 293 K):δ 7.16 (2 H, d), 6.62 (1 H, t), 3.89 (3 H, s), 3.81 (6 H, s).

The obtained Compound 7-1 (2.0 g, 10.2 mmol) and NaBH₄ (2.12 g, 56.1 mmol) were put into THF (75 mL), and MeOH (20 mL) was slowly added for 1 hour while the resultant mixture was refluxed. After the MeOH addition, refluxing was further performed for 1 hour, the temperature was reduced to room temperature, and then 1M HCl was added to the mixture cooled to room temperature to adjust pH to 7. Subsequently, an organic layer was extracted using EtOAc (200 mL, four times), and then dehydrated with Na₂SO₄ (10 g) to remove residual water therein. In addition, the organic layer was concentrated using an aspirator (25° C., 20˜500 mmHg), thereby obtaining Compound 7-2 (1.22 g, 94%), and the following procedure was performed without a separate separating procedure. 1 H NMR (CDCl₃, 300 MHz, 293 K):δ 6.51 (2 H, d), 6.37 (1 H, t), 4.61 (2 H, s), 3.78 (6 H, s).

A mixture prepared by dissolving the obtained Compound 7-2 (1.0 g, 5.95 mmol) in dichloromethane (50 mL) and adding pyridinium chlorochromate (3.85 g, 17.85 mmol) at room temperature was stirred at room temperature for 3 hours. After 3 hours, 2 g of silica was added to the mixture, and dichloromethane was removed using an aspirator (25° C., 20˜500 mmHg). A dichloromethane-removed silica solid was filtered, and washed with a 10% EtOAc/Hexane solution several times, and then a solvent was removed from the solution collected by a filter using an aspirator, thereby obtaining a colorless liquid, Compound 7-3 (920 mg, 93%), and the following procedure was performed without a separate separating procedure. 1 H NMR (CDCl₃, 300 MHz, 293 K):δ 9.90 (1 H, s), 7.00 (2 H, d), 6.69 (1 H, t), 3.84 (6 H, s).

A mixture prepared by dissolving Compound 7-3 (500 mg, 3.0 mmol) in chloroform (10 mL) and adding 1,3-dibromo-5,5-dimethylhydantoin (430 mg, 1.5 mmol) at 0° C. was stirred at room temperature for 3 hours, and then H₂O (30 mL) was added to extract an organic layer. The extracted organic layer was dried with Na₂SO₄ (5 g), and concentrated using an aspirator (25° C., 20˜500 mmHg), thereby obtaining a white solid, Compound 8 (700 mg, 95%), and the following procedures were performed without a separate separating procedure. 1 H NMR (CDCl₃, 300 MHz, 293 K):δ 10.41 (1 H, s), 7.04 (1 H, d), 6.71 (1 H, d), 3.91 (3 H, s), 3.85 (3 H, s). 13 C NMR (75 MHz, CDCl₃):δ 192.1, 160.0, 157.1, 134.7, 109.1, 105.9, 103.4, 56.6, 55.8.

Step 1-3: Synthesis of 2-(1,3-dioxolan-2-yl)-4,6-dimethoxybenzaldehyde

To synthesize Compound 9 of Reaction Formula 1, 2-(1,3-dioxolan-2-yl)-4,6-dimethoxybenzaldehyde, Compound 8 (500 mg, 2.04 mmol) obtained in Step 1-2 was dissolved in toluene (20 mL). In addition, ethylene glycol (190 μL, 3.06 mmol) and p-toluenesulfonic acid monohydrate (39 mg, 0.21 mmol) were added, and then refluxing was performed in the Dean-Stark apparatus for 24 hours. After 24 hours, a reaction vessel was reduced to room temperature, 5 mL of a saturated KOH-EtOH solution was added, the resultant mixture was stirred at room temperature for 30 minutes, and then 50 mL of H₂O was added. Afterward, an organic layer was extracted with EtOAc (50 mL), dehydrated with Na₂SO₄ (5 g) to remove residual water, and then concentrated using an aspirator. In addition, through column chromatography (diameter: 3 cm, height: 15 cm; developing solvent: 10% EtOAc/Hexane) using silica gel, a white solid, Compound 8-1 (554 mg, 94%), was obtained. 1 H NMR (CDCl₃, 300 MHz, 293 K):δ 16.75 (1 H, d), 6.44 (1 H, d), 6.06 (1 H, s), 4.12-3.97 (4 H, m), 3.80 (3 H, s), 3.76 (3 H, s). 13 C NMR(CDCl₃, 75 MHz, 293 K):δ 159.8, 156.6, 138.3, 103.4, 102.4, 100.5, 65.3, 56.3, 55.5.

Compound 8-1 (458 mg, 1.58 mmol) was dissolved in a THF (10 mL) solution and decreased in temperature to −78° C., n-BuLi (1.6 M in hexane, 1.09 mL, 1.74 mmol) was slowly added, and then the resultant mixture was stirred at room temperature for 1 hour. After 1 hour, the temperature was reduced again to 0° C., the mixture to which DMF (370 μL, 7.42 mmol) was slowly added was further stirred at the same temperature for 1 hour, and then NH₄Cl (2 mL) was added to terminate the reaction. The reaction-terminated mixture was treated with EtOAc (20 mL) and H₂O (20 mL) to extract an organic layer, and the obtained organic layer was dehydrated with Na₂SO₄ (5 g) to remove residual water and concentrated using an aspirator (25° C., 20˜500 mmHg), thereby obtaining Compound 9 (443 mg, 82%).

Compound 9 obtained by concentration as described above was prepared to perform the following procedures without a separate separating procedure. 1 H NMR (CDCl₃, 300 MHz, 293 K):δ 10.36 (1 H, s), 6.84 (1 H, d), 6.50 (1 H, s), 6.37 (1 H, d), 4.00-3.95 (4 H, m), 3.79 (6 H, s). 13 C NMR(CDCl₃, 75 MHz, 293 K):δ 189.5, 164.9, 164.8, 142.4, 116.6, 103.6, 99.6, 98.2, 65.2, 55.9, 55.5.

Step 1-4: Synthesis of (E)-3-(2-(1,3-dioxolan-2-yl)-4,6-dimethoxyphenyl)-1-(6-(2-hydroxyethylamino)naphthalene-2-yl)prop-2-en-1-one

To synthesize Compound 10 of Reaction Formula 1, (E)-3-(2-(1,3-dioxolan-2-yl) -4,6-dimethoxyphenyl)-1-(6-(2-hydroxyethylamino)naphthalene-2-yl)prop-2-en-1-one, Compound 6 (230 mg, 1.0 mmol) obtained in Step 1-1 and Compound 9 (477 mg, 2.0 mmol) obtained in Step 1-3 were dissolved in EtOH (5 mL). In addition, a catalytic amount of NaOH (23 mg) was added at room temperature, a temperature was increased, refluxing was performed for 3 hours, the temperature was reduced again to room temperature, and then EtOH was removed using an aspirator. Dichloromethane (30 mL) and H₂O (10 mL) were added to the mixture from which EtOH was removed to extract an organic layer, and the organic layer obtained by extraction as described above was dehydrated with Na₂SO₄ (5 g) to remove residual water and then concentrated using an aspirator. Finally, through column chromatography (diameter: 2 cm, height: 15 cm; developing solvent: 50% EtOAc/Hexane) using silica gel, a solid, Compound 10 (383 mg, 85%), was obtained.

1 H NMR (CDCl₃, 300 MHz, 293 K):δ 8.34 (1 H, s), 9.09 (1 H, d), 7.96 (1 H, dd), 7.85 (1 H, d), 7.64 (1 H, d), 7.56 (1 H, d), 6.92 (1 H, d), 6.86 (1 H, dd), 6.75 (1 H, d), 6.52 (1 H, d), 6.04 (1 H, s), 4.22-4.16 (2 H, m), 4.14-4.04 (2 H, m), 3.94 -3.89 (5 H, m), 3.87 (3 H, s), 3.37 (2 H, t). 13 C NMR(CDCl₃, 75 MHz, 293 K):δ 190.0, 161.7, 160.9, 148.3, 139.5, 138.0, 136.8, 132.3, 131.0, 130.6, 126.4, 126.3, 125.9, 125.6, 118.8, 117.2, 104.2, 103.1, 101.4, 99.6, 65.6, 61.2, 56.0, 55.7, 45.9.

Step 1-5: Synthesis of (E)-2-(3-(6-(2-hydroxyethylamino) naphthalene-2-yl)-3 -oxoprop-1-enyl)-3,5-dimethoxybenzaldehyde

Finally, to synthesize Compound 2 of Reaction Formula 1, (E)-2-(3-(6-(2-hydroxyethylamino)naphthalene-2-yl)-3-oxoprop-1-enyl)-3,5-dimethoxybenzaldehyde, a mixture prepared by dissolving Compound 10 (383 mg, 0.85 mmol) obtained in Step 1-4 in CH₃CN (7.5 mL) was decreased in temperature to 0° C., and then HCl (0.5 mL) was slowly added. In addition, the resultant mixture was stirred at the same temperature for 5 minutes, 10 ml of a saturated NaHCO₃ solution was added to terminate the reaction, and an organic layer was extracted with dichloromethane (30 mL), dehydrated with Na₂SO₄ (3 g) to remove residual water and concentrated using an aspirator. Afterward, through column chromatography (diameter: 2 cm, height: 15 cm; developing solvent: 50% EtOAc/Hexane) using silica gel, a solid, Compound 2 (300 mg, 87%), was finally obtained. 1 H NMR (CDCl₃, 300 MHz, 293 K):δ 10.33 (1 H, s), 8.32 (1 H, s), 8.23 (1 H, d), 7.97 (1 H, d), 7.69 (1 H, d), 7.60 (1 H, d), 7.35 (1 H, d), 7.07 (1 H, d), 6.91 (1 H, d), 6.80 (1 H, s), 6.72 (1 H, s), 3.95-3.90 (8 H, m), 3.42 (2 H, t). 13 C NMR(CDCl₃, 75 MHz, 293 K):δ 191.7, 189.1, 161.6, 160.4, 148.5, 138.2, 137.4, 135.1, 131.8, 131.2, 130.7, 130.0, 126.5, 126.4, 125.5, 122.2, 118.9, 104.3, 103.5, 61.3, 56.3, 56.0, 45.8. HRMS: m/z calcd for C₂₄H₂₃NO₅: 405.1576, found 405.1574.

### Synthesis Example 2

### Synthesis and Structural Analysis of Compound 3

The inventors synthesized Compound 3 of Formula 3 according to the pathway represented by Reaction Formula 2.

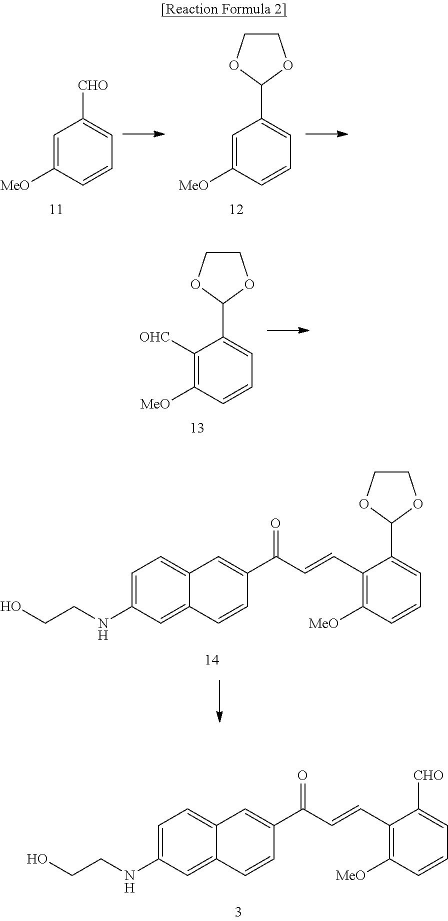 [SMILES: COc1cccc(C=O)c1 ; COc1cccc(C2OCCO2)c1 ; COc1cccc(C2OCCO2)c1C=O ; COc1cccc(C2OCCO2)c1/C=C/C(=O)c1ccc2cc(NCCO)ccc2c1 ; COc1cccc(C=O)c1/C=C/C(=O)c1ccc2cc(NCCO)ccc2c1]

Step 2-1: Synthesis of 2-(3-methoxyphenyl)-1,3-dioxolane

To synthesize Compound 12 of Reaction Formula 2, 2-(3-methoxyphenyl)-1, 3-dioxolane, Compound 11 (1.0 g, 7.34 mmol), which was a starting material for synthesis, was dissolved in toluene (20 mL). In addition, ethylene glycol (611 μL, 11.02 mmol) and p-toluenesulfonic acid monohydrate (140 mg, 0.734 mmol) were added, and the resultant mixture was refluxed in the Dean-Stark apparatus for 24 hours. After 24 hours, a reaction vessel was cooled to room temperature, 5 mL of a saturated KOH-EtOH solution was added, and the resultant mixture was stirred at room temperature for 30 minutes. 50 mL of H₂O was added, and an organic layer was extracted using EtOAc (50 mL). The organic layer obtained by extraction was dehydrated with Na₂SO₄ (5 g) to remove residual water and concentrated using an aspirator, and then through column chromatography (diameter: 3 cm, height: 15 cm; developing solvent: 10% EtOAc/Hexane) using silica gel, Compound 12 (1.21 g, 92%) was obtained. 1 H NMR (CDCl₃, 300 MHz, 293 K):δ 7.28 (1 H, t), 7.10-7.05 (2 H, m), 6.94-6.90 (1 H, m), 5.80 (1 H, s), 4.14-3.98 (4 H, m), 3.81 (3 H, s). 13 C NMR(CDCl₃, 75 MHz, 293 K):δ 159.9, 139.7, 129.6, 119.0, 115.2, 111.6, 103.7, 65.4, 55.4.

Step 2-2: Synthesis of 2-(1,3-dioxolan-2-yl)-6-methoxybenzaldehyde

To synthesize Compound 13 of Reaction Formula 2, 2-(1,3-dioxolan-2-yl)-6-methoxybenzaldehyde, Compound 12 (930 mg, 5.16 mmol) as a starting material for synthesis was dissolved in 30 mL of cyclohexane, and decreased in temperature to 0 ° C. using ice water. In addition, n-BuLi (1.6 M in hexane, 3.225 mL, 5.16 mmol) was added and reacted at room temperature for 30 minutes, DMF (0.803 μL, 10.32 mmol) was added, and the resultant mixture was stirred for 1 hour. After stirring, an organic layer was extracted using 5 mL of saturated saline and 20 mL of H₂O and EtOAc (50 mL), the organic layer obtained by extraction was dehydrated with anhydrous sodium sulfate (5 g) to remove residual water in the organic layer and concentrated using an aspirator, thereby obtaining a light yellow liquid, Compound 13 (773 mg, 72%). The obtained Compound 13 was used in the following reactions without a separate separating procedure. 1 H NMR (CDCl₃, 300 MHz, 293 K):δ 10.60 (1 H, s), 7.50 (1 H, t), 7.36 (1 H, d), 7.00 (1 H, dd), 6.52 (1 H, s), 4.08-4.05 (4 H, m), 3.91 (3 H, s). 13 C NMR(CDCl₃, 75 MHz, 293 K):δ 191.9, 162.6, 140.3, 134.9, 123.5, 118.7, 112.6, 100.1, 65.5, 56.2.

Step 2-3: Synthesis of (E)-3-(2-(1,3-dioxolan-2-yl)-6-methoxyphenyl)-1-(6-(2-hydroxyethylamino)naphthalene-2-yl)prop-2-en-1 -one

To synthesize Compound 14 of Reaction Formula 2, (E)-3-(2-(1,3-dioxolan-2-yl)-6-methoxyphenyl)-1-(6-(2-hydroxyethylamino) naphthalene-2-yl)prop-2-en-1-one, Compound 13 (95 mg, 0.456 mmol) obtained from Step 2-2 and Compound 6 (52 mg, 0.228 mmol) obtained from Step 1-1 in Synthesis Example 1 were used as starting materials for synthesis, and synthesis was performed by the same method as

Step 1-4 in Synthesis Example 1, thereby obtaining Compound 14 (70 mg, 74%). 1 H NMR (CDCl₃, 300 MHz, 293 K):δ 8.38 (1 H, s), 8.10 (1 H, d), 8.00 (1 H, d), 7.84 (1 H, d), 7.68 (1 H, d), 7.60 (1 H, d), 7.41-7.34 (2 H, m), 7.00-6.90 (2 H, m), 6.81 (1 H, s), 6.01 (1 H, s), 4.51 (1 H, br), 4.24-4.16 (2 H, m), 4.12-4.02 (2 H, m), 3.92 (3 H, s), 3.41 (2 H, t), 1.98 (1 H, br). 13 C NMR(CDCl₃, 75 MHz, 293 K):δ 190.4, 158.9, 148.3, 138.1, 137.9, 136.9, 142.2, 131.2, 130.8, 130.2, 128.6, 126.5, 126.4, 125.7, 124.6, 119.1, 118.8, 111.9, 104.3, 101.7, 65.7, 61.3, 56.1, 45.9.

Step 2-4: Synthesis of (E)-2-(3-(6-(2-hydroxyethylamino)naphthalene-2-yl)-3-oxoprop-1-enyl)-3-methoxybenzaldehyde

Finally, Compound 3 of Reaction Formula 2, (E)-2-(3-(6-(2-hydroxyethylamino)naphthalene-2-yl)-3-oxoprop-1-enyl)-3 -methoxybenzaldehyde, was synthesized. Compound 14 (70 mg, 0.167 mmol) obtained from Step 2-3 was used as a starting material, and the synthesis was performed by the same method as shown in Step 1-5 of Synthesis Example 1, thereby obtaining Compound 3 (52 mg, 83%). 1 H NMR (CDCl₃, 300 MHz, 293 K):δ 10.32 (1 H, s), 8.33 (1 H, s), 8.25 (1 H, d), 8.00 (1 H, d), 7.69 (1 H, d), 7.63-7.56 (2 H, m), 7.51-7.36 (1 H, m), 7.16 (1 H, d), 6.91 (1 H, d), 6.81 (1 H, s), 4.50 (1 H, br), 3.95-3.87 (5 H, m), 3.43 (2 H, t), 2.02 (1 H, br). 13 C NMR(CDCl₃, 75 MHz, 293 K):δ 192.1, 189.0, 158.8, 148.5, 138.3, 136.4, 135.4, 131.7, 131.5, 131.3, 130.9, 130.3, 128.4, 126.6, 126.4, 125.5, 121.4, 119.0, 115.7, 104.2, 61.2, 56.3, 45.8. HRMS (FAB): m/z calcd for C₂₃H₂₁NO₄: 375.1471, found 375.1469.

### Synthesis Example 3

### Synthesis and Structural Analysis of Compound 4

The inventors synthesized Compound 4 of Formula 4 according to the pathway represented of Reaction Formula 3.

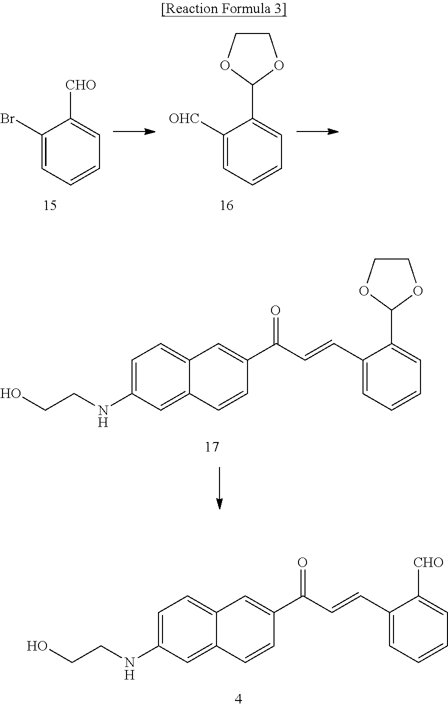 [SMILES: O=Cc1ccccc1Br ; O=Cc1ccccc1C1OCCO1 ; O=C(/C=C/c1ccccc1C1OCCO1)c1ccc2cc(NCCO)ccc2c1 ; O=Cc1ccccc1/C=C/C(=O)c1ccc2cc(NCCO)ccc2c1]

Step 3-1: Synthesis of 2-(1,3-dioxolan-2-yl)benzaldehyde

To synthesize Compound 16 of Reaction Formula 3, 2-(1,3-dioxolan-2-yl)benzaldehyde, Compound 15 (1.0 g, 5.4 mmol) as a starting material for synthesis was dissolved in toluene (20 mL). In addition, ethylene glycol (0.5 mL, 8.1 mmol) and p-toluenesulfonic acid monohydrate (102 mg, 0.54 mmol) were added, and refluxing was performed in the Dean-Stark apparatus for 24 hours. After 24 hours, a reaction vessel was cooled to room temperature, 5 mL of a saturated KOH-EtOH solution was added, and then the resultant mixture was stirred at room temperature for 30 minutes and mixed with 50 mL of water. From the above mixture, an organic layer was extracted with EtOAc (50 mL). The obtained organic layer was dehydrated with Na₂SO₄ (5 g) to remove residual water therein, and concentrated using an aspirator. In addition, through column chromatography (diameter: 3 cm, height: 15 cm; developing solvent: 5% EtOAc/Hexane) using silica gel, Compound 15-1 (1.1 mg, 89%) was obtained. 1 H NMR (CDCl₃, 300 MHz, 293 K):δ 7.62-7.55 (2 H, m), 7.31 (1 H, dt), 7.18 (1 H, dt), 6.11 (1 H, s), 4.02-4.17 (4 H, m). 13 C NMR(CDCl₃, 75 MHz, 293 K):δ 136.9, 133.2, 130.8, 128.1, 127.6, 123.2, 102.8, 65.7.

Here, the synthesized compound 15-1 (230 mg, 1.0 mmol) was dissolved in 5 mL of THF, decreased in temperature to −78° C. using dry ice-acetone, mixed with n-BuLi (1.6 M in hexane, 0.94 mL, 1.5 mmol), and then stirred at the same temperature for 1 hour. After 1 hour, DMF (117 μL, 1.5 mmol) was added, and the resultant mixture was gradually heated and stirred at 0° C. for 1 hour, and then treated with 2 mL of a saturated NH₄Cl solution to terminate the reaction. Subsequently, extraction was performed using 10 mL of H₂O and 10 mL of EtOAc. The organic layer obtained by extraction was dehydrated with Na₂SO₄ (5 g) to remove residual water therein and concentrated using an aspirator, thereby obtaining a light yellow liquid, Compound 16 (147 mg, 82%), and then the compound was used in the following reactions without a separate separating procedure. 1 H NMR (CDCl₃, 300 MHz, 293 K):δ 10.42 (1 H, s), 7.94 (1 H, dd), 7.73 (1 H, dd), 7.6 (1 H, dt), 7.54 (1 H, dd), 6.42 (1 H, s), 4.17-4.12 (4 H, m). 13 C NMR(CDCl₃, 75 MHz, 293 K):δ 192.0, 139.3, 134.7, 133.8, 130.4, 129.7, 127.2, 101.3, 65.6.

Step 3-2: Synthesis of (E)-3-(2-(1,3-dioxolan-2-yl)phenyl)-1-(6-(2-hydroxyethylamino)naphthalene-2-yl)prop-2-en-1-one

Compound 17 of Reaction Formula 3, (E)-3-(2-(1,3-dioxolan-2-yl)phenyl)-1- (6-(2-hydroxyethylamino)naphthalene-2-yl)prop-2-en-1-one, was synthesized. Compound 17 (61 mg, 72%) was obtained by the same method as shown in Step 1-4 of Synthesis Example 1 using Compound 16 (117 mg, 0.654 mmol) obtained from Step 3-1 and Compound 6 (50mg, 0.218 mmol) obtained from Step 1-1 of Synthesis Example 1 as starting materials for synthesis. 1 H NMR (CDCl₃, 300 MHz, 293 K):δ 8.39 (1 H, s), 8.27 (1 H, d), 8.00 (1 H, dd), 7.77-7.80 (1 H, m), 7.72 (1 H, d), 7.56-7.68 (3 H, m), 7.43-7.46 (2 H, m), 6.93 (1 H, dd), 6.83 (1 H, d), 6.09 (1 H, s), 4.50 (1 H, br), 4.18-4.22 (2 H, m), 4.05-4.10 (2 H, m), 3.91-3.96 (2 H, m), 3.44 (2 H, br), 1.80 (1 H, t). 13 C NMR(CDCl₃, 75 MHz, 293 K):δ 189.8, 148.4, 141.0, 138.1, 136.6, 134.9, 132.0, 131.2, 130.7, 130.0, 129.6, 127.3, 127.2, 126.5, 125.6, 124.9, 118.9, 104.3, 102.2, 65.7, 61.3, 45.8.

Step 3-3: Synthesis of (E)-2-(3-(6-(2-hydroxyethylamino)naphthalene-2-yl)-3-oxoprop-1-enyl)benzaldehyde

Finally, Compound 4 of Reaction Formula 3, (E)-2-(3-(6-(2-hydroxyethylamino)naphthalene-2-yl)-3-oxoprop-1-enyl)benzaldehyde, was synthesized. Compound 4 (42 mg, 78%) was obtained by the same method as shown in Step 1-5 of Synthesis Example 1 using Compound 17 (61 mg, 0.156 mmol) obtained from Step 3-2 as a starting material. 1 H NMR (CDCl₃, 300 MHz, 293 K):δ 10.4 (1 H, s), 8.55 (1 H, d), 8.43 (1 H, s), 8.01 (1 H, dd), 7.92 (1 H, dd), 7.81-7.77 (2 H, m), 7.68-7.65 (2 H, m), 7.58 (1 H, dd), 7.50 (1 H, d), 6.95 (1 H, dd), 6.85 (1 H, d), 4.51 (1 H, br), 3.94 (2 H, t), 3.45 (2 H, t), 1.71 (1 H, br). 13 C NMR(CDCl₃, 75 MHz, 293 K):δ 191.7, 189.4, 148.3, 140.0, 138.1, 137.9, 134.3, 133.9, 131.7, 131.4, 131.1, 130.8, 129.8, 128.2, 127.7, 126.4, 126.2, 125.4, 118.8, 104.1, 61.1, 45.6. HRMS (FAB): m/z calcd for C₂₂H₁₉NO₃: 345.1365, found 345.1365.

### Example 1

### Confirmation of Fluorescence Change Due to Reaction Between Hydrogen Sulfide and Compound 2

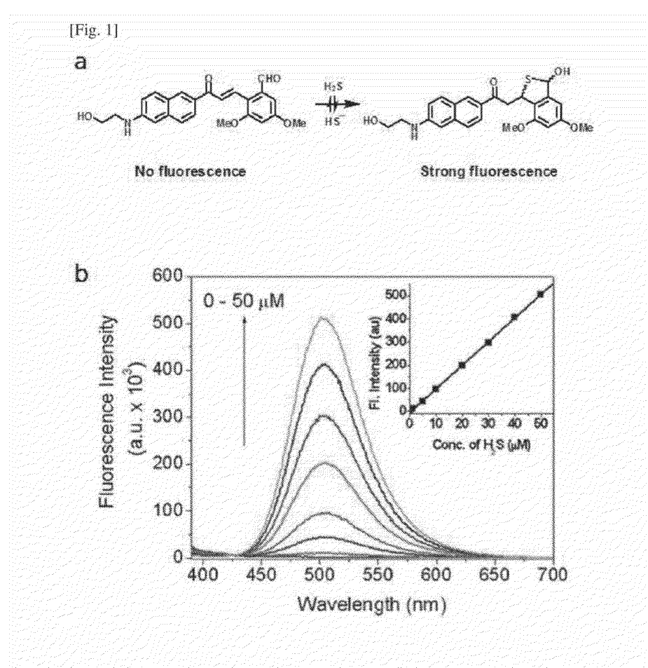

A mechanism of a fluorescence turn-on phenomenon according to a reaction between Compound 2 and hydrogen sulfide is shown in FIG. 1a, an α-β unsaturated carbonyl group of Compound 2 reacted with hydrogen sulfide to induce a ring-shape in a chemical reaction. A product generated by the chemical reaction exhibited strong fluorescence, and when an excitation wavelength was 375 nm, a fluorescence emission wavelength was detected to be 510 nm.

Therefore, to observe the fluorescence change of Compound 2 due to hydrogen sulfide, a fluorescence graph of Compound 2 was measured in a buffer (pH 7.4, 10 mM HEPES buffer). For fluorescence spectra analysis, a photon technical international fluorescence system manufactured by PTI was used, as a cell providing Compound 2 to each instrument, a standard quartz cell having a thickness of 1 cm was used. First, Compound 2 (10 μM) was treated with hydrogen sulfide at a concentration of 0 to 50 μM, and after 5 minutes, a fluorescence graph was checked.

As a result, as shown in FIG. 1b, since the amount of a fluorescent reaction product was increased by increasing a concentration of hydrogen sulfide, it was confirmed that a fluorescence intensity was increased (vertical axis: fluorescence intensity, horizontal axis: wavelength). An inner graph shows the fluorescence intensity at an emission wavelength of 510 nm, and it can be seen that the fluorescence values are plotted in a linear shape according to the concentration of hydrogen sulfide.

### Example 2

### Observation of Fluorescence Changes of Compound 2 and Hydrogen Sulfide Over Time

To observe the fluorescence change of Compound 2 over time due to hydrogen sulfide, Compound 2 (10 μM) was treated with 100 μM of hydrogen sulfide (using the same buffer as used in Example 1), and a graph of fluorescence over time was analyzed. When an excitation wavelength was 375 nm, a fluorescence emission wavelength was detected to be 510 nm.

As a result, as shown in FIG. 2, it was seen that Compound 2 approached the maximum fluorescence level within 5 minutes, and fluorescence emission was saturated in about 10 minutes (vertical axis: fluorescence intensity, horizontal axis: wavelength). An inner graph shows the fluorescence intensity at an emission wavelength of 510 nm.

### Example 3

### Observation of Fluorescence Changes of Compound 2 According to Reaction Between Hydrogen Sulfide and Biological Sulfide

To confirm the hydrogen sulfide selectivity of Compound 2 under hydrogen sulfide and biological sulfide conditions, the fluorescence change of Compound 2 (10 μM) was observed under biological sulfide conditions (Na₂S (100 μM), the same material as H₂S), glutathione (GSH, 10 mM), cysteine (Cys, 200 μM), and homocysteine (Hcy, 50 μM) (using the same buffer as used in Example 1)). Here, an excitation wavelength was 375 nm, and a fluorescence emission wavelength was detected to be 510 nm.

As a result, as shown in FIG. 3, after 30 minutes, it was confirmed that Compound 2 showed a sufficient fluorescence turn-on phenomenon only in a reaction with Na₂S (the same as H₂S) (vertical axis: fluorescence intensity, horizontal axis: wavelength).

From the above, it can be seen that Compound 2 can selectively sense H₂S under a condition of various biological sulfides.

### Example 4

### Observation of Fluorescence Change of Compound 2 According to Reaction with Various Types of Biological Substances

To observe fluorescence changes according to reactions between various types of biological substances and Compound 2, Compound 2 (10 μM) was reacted with a biologically-active substance (an amino acid (Ala, Glu, Lys, or Met), lipoic acid, an anion (NO²⁻, SO₄²⁻, S₂O₃²⁻, SCN⁻, or I⁻), and active oxygen (H₂O₂). A buffer used in the experiment was the same as used in Example 1, and the concentration of each biologically-active substance was 100 μM. Each biologically-active substance was added, and after about 30 minutes, an excitation wavelength was 375 nm, and a fluorescence emission wavelength was detected to be 510 nm.

As a result, as shown in FIG. 4, it can be confirmed that Compound 2 only reacts with hydrogen sulfide (H₂S), and thus selectively exhibits a fluorescence turn-on phenomenon (vertical axis: fluorescence intensity, horizontal axis: type of biologically-active substance).

### Example 5

### Analysis of Hydrogen Sulfide Sensitivity of Compound 2 by Fluorescence Change

To observe the hydrogen sulfide sensitivity of Compound 2 based on fluorescence change, an amount of Na₂S (the same as H₂S) in Compound 2 (10 μM) was reduced. A buffer used in the experiment was the same as used in Example 1, 50 nM of Na₂S was added, an excitation wavelength was 375 nm, and a fluorescence emission wavelength was detected to be 510 nm.

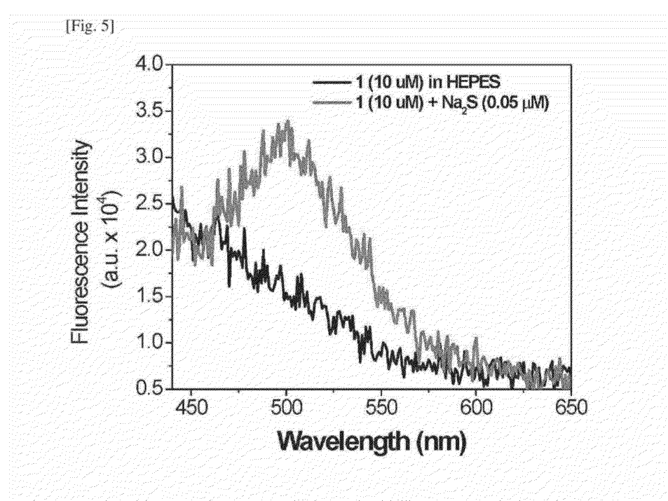

As a result, about 5 minutes after Na₂S was added, the fluorescence turn-on with a signal to noise ratio of 3 or higher was observed, and as shown in FIG. 5, it can be seen that even at a low concentration of 50 nM, the fluorescence of Compound 2 can be observed (vertical axis: fluorescence intensity, horizontal axis: wavelength).

### Example 6

### Fluorescence Changes Of Compound 2 Due to Hydrogen Sulfide Under Various Acidity Conditions

To observe fluorescence changes of Compound 2 due to hydrogen sulfide under various acidity (pH) conditions, the fluorescence changes were examined when Compound 2 (10 μM) bound to H₂S under various acidity conditions (pH 5˜9). In other words, 100 μM of H₂S reacted to Compound 2 at pH 5, 6, 7, 8, or 9, and then 5 minutes later, a fluorescence intensity was measured. Here, an excitation wavelength was 375 nm, and a fluorescence emission wavelength was detected to be 510 nm.

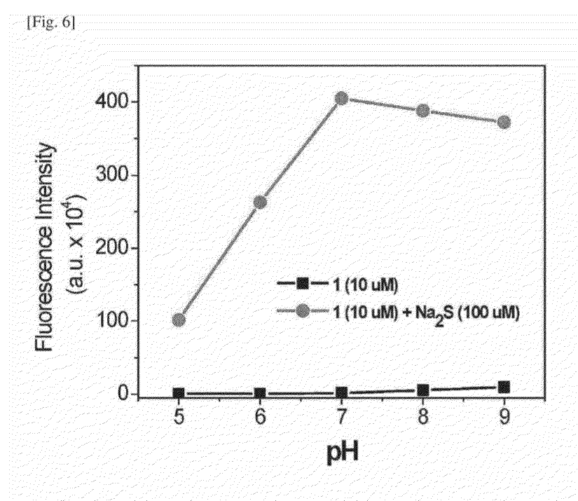

As a result, as shown in FIG. 6, it can be confirmed that the sharpest increase in fluorescence was shown at neutral pH, and a relatively less increase in fluorescence was shown at acidic pH (vertical axis: fluorescence intensity, horizontal axis: pH).

### Example 7

### Cell Imaging Using One-Photon and Two-Photon Fluorescence Microscopes by Treatment with Compound 2

To observe a fluorescence change according to the treatment with Compound 2 through cell imaging using one-photon and two-photon fluorescence microscopes, Compound 2 (10 μM) was treated with human cervical carcinoma cells (HeLa cells). The HeLa cells were cultured in a Dulbecco's modified eagles medium (DMEM, Hyclone) containing 10% fetal bovine serum (Hyclone) and penicillin-streptomycin (Hyclone) with 5% carbon dioxide at an ambient temperature of 37° C. to a cell density of about 20,000 cells/cm², and used in the experiment. The used one-photon fluorescence microscope is an LSM710 confocal microscope manufactured by Carl Ziess, and the two-photon fluorescence microscope is a Chameleon Ultra model having a Ti-sapphire laser, which is manufactured by Coherent. A lens used in the two-photon fluorescence microscope is an XLUMPLFNM, NA 1.0 model manufactured by Olympus, and a wavelength and laser power of the two-photon fluorescence microscope are 880 nm and 15 mW, respectively.

Sets of the experiment are as follows: (1) a control set which has not been treated; (2) a set of cells treated only with a probe (10 μM) of Compound 2 (Cpd 2) and cultured for 30 minutes; (3) a set of cells pre-treated with GSH (300 μM), cultured for 30 minutes, treated with a probe (10 μM) of Compound 2 (Cpd 2), and further cultured for 30 minutes; (4) a set of cells pre-treated with Cys (300 μM), cultured for 30 minutes, treated with a probe (10 μM) of Compound 2 (Cpd 2), and further cultured for 30 minutes; (5) a set of cells pre-treated with Na₂S (300 μM), cultured for 30 minutes, treated with a probe (10 μM) of Compound 2 (Cpd 2), and further cultured for 30 minutes; and (6) a set of cells pre-treated with phorbol 12-myristate 13-acetate (PMA; 50 μM), cultured for 30 minutes, treated with a probe (10 μM) of Compound 2 (Cpd 2), and further cultured for 30 minutes.

Observation results are shown in FIG. 7, a one-photon fluorescence microscope result is shown in an upper image of FIG. 7a, and a two-photon fluorescence microscope result is shown in a lower image of FIG. 7a. Also, a scale bar of the one-photon fluorescence microscope is 60 μm, and a scale bar of the two-photon fluorescence microscope is 30 μm. Since the set (1) was not treated with Compound 2, no image was observed using the one-photon fluorescence microscope, and pale auto-fluorescence was observed using the two-photon fluorescence microscope. The set (2) showed an increase in fluorescence since Compound 2 sensed H₂S in the cells. The sets (3) and (4) showed stronger fluorescence change than the set (2) only treated with Compound 2, due to an increased amount of H₂5 in the cells resulting from pre-treated GSH and Cys. The set (5) showed stronger fluorescence than the sets (2) to (4) since H₂5 was pre-treated. The set (6) showed an increase in fluorescence by decreasing the amount of hydrogen sulfide (H₂S) in the cells due to PMA. Averages of the fluorescence intensities for these sets are shown in FIGS. 7b and 7c (vertical axis: fluorescence intensity, horizontal axis: set).

From the above results, it can be seen that Compound 2 easily permeates into cells, and reacts with hydrogen sulfide in the cells to produce fluorescence change.

### Example 8

### Tissue Imaging of Compound 2 Treated Mouse Using Two-Photon Fluorescence Microscope

Tissue imaging per each organ of a Compound 2 treated mouse was performed using a two-photon fluorescence microscope. That is, the distribution of hydrogen sulfide (H₂S) in each organ (the brain, kidney, liver, spleen or lung) of a mouse was confirmed using Compound 2. To this end, a set (1′) was prepared by injecting Compound 2 into the abdominal cavity of a live mouse and extracting an organ, and a set (2′) was prepared by extracting each organ of a mouse and immersing the organ in a solution of Compound 2. The mouse used in the experiment was a C57BL6 type (SAMTAKO Corp.), which is five weeks old. More particularly, for the set (1′), 20 μL of a 10 mM solution of Compound 2, which had been taken and diluted in 280 μL of a PBS (100 mM, pH 7.4) buffer, was injected into the abdominal cavity of a mouse twice a day for a total of 5 days, and then each organ was extracted. The extracted organ was frozen in dry ice for 5 minutes, crushed into smaller pieces with a hammer, and cut to a thickness of 16 μm using a section machine (Cryostat machine, Leica, CM3000 model). Each piece of the organ tissue was put into an OCT complex (10% w/w polyvinyl alcohol, 25% w/w polyethylene glycol, 85.5% w/w inactive species) to fix, put on a specimen block (Paul Marienfeld GMbH & Co.), treated with 4% paraformaldehyde (PFA), and stored for 10 minutes. Subsequently, the resultant sample was washed three times with a PBS buffer, covered with a mount solution (Gel Mount, BIOMEDA), and then imaged using a two-photon fluorescence microscope, which is the same as used in Example 7. However, an excitation wavelength and laser power of the two-photon fluorescence microscope were 880 nm and 40 mW, respectively. Also, for the set (2′), first, each organ of a mouse was extracted and immersed in a solution (10 μM) of Compound 2 for 10 minutes, and then a sample prepared as described above was imaged by the same method used for the set (1′).

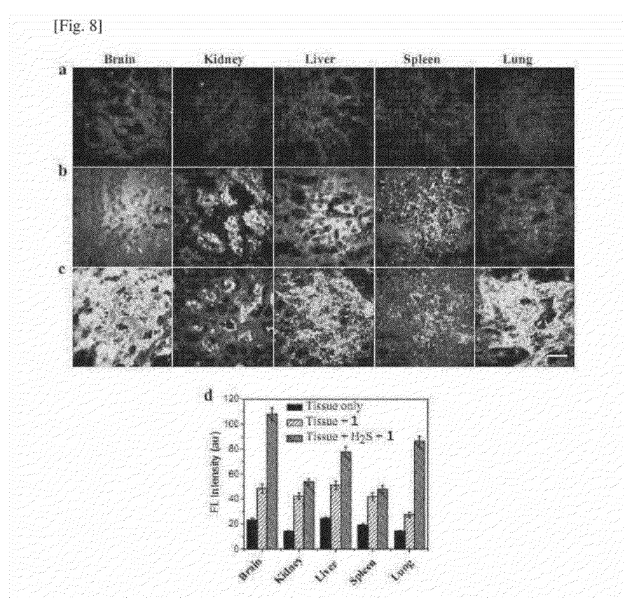

Results of the mouse tissue imaging are shown in FIG. 8. FIG. 8a is a two-photon fluorescent image of each organ tissue not treated with Compound 2 as the control, which exhibits a very small auto-fluorescence value. FIG. 8b shows the result for the set (1′) showing that signals are increased in the brain, kidney, liver, spleen and lung. Since Compound 2 was administered into the living mouse by abdominal injection, it can be seen that Compound 2 was permeated throughout the organ, particularly, into the brain so as to sense hydrogen sulfide in the brain. FIG. 8c shows the result for the set (b′) showing that strong fluorescence changes are shown in the brain, liver and lung, and a degree of the distribution of hydrogen sulfide in each organ can be confirmed. In FIGS. 8a, 8b and 8c, a scale bar is 30 μm, and in FIG. 8d, the average value of fluorescence intensities of each organ is shown. Here, a vertical axis represents the fluorescence intensity in each tissue, and a horizontal axis represents each organ.

### Example 9

### Tissue Imaging of Compound 2-Treated Fish Using Two-Photon Fluorescence Microscope

A tissue of each organ of a Compound 2-treated zebrafish was imaged using a two-photon fluorescence microscope. That is, an experiment for confirming the distribution of hydrogen sulfide (H₂5) in a zebrafish was performed by culturing the fish in an environment having Compound 2 and extracting the organ. A 6-month-old zebrafish was used, and the experiment was designed with a total of two sets. For the set (1″), a zebrafish was cultured in E3 media (15 mM NaCl, 0.5 mM KCl, 1 mM MgSO₄, 1 mM CaCl₂, 0.15 mM KH₂PO₄, 0.05 mM Na₂HPO₄, 0.7 mM NaHCO₃, pH 7.4) containing Compound 2 at a concentration of 100 μM, cultured at 27° C. for about 20 minutes and washed several times with fresh E3 media, and then each organ (9 organs including the brain, swim bladder, eyes, gills, heart, spleen, liver, and kidney) was extracted and observed using a two-photon fluorescence microscope, which is the same as used in Example 7. Each organ was fixed with 7% methyl cellulose. However, here, an excitation wavelength and laser power of the two-photon fluorescence microscope were 880 nm and 40˜60 mW, respectively. For the set (2″), the zebrafish, which has been cultured with Compound 2 in the set (1″), was washed several times with E3 media, and further cultured in a hydrogen sulfide solution. Here, the hydrogen sulfide had a concentration of 200 μM, and after about 20-minute culturing, imaging was performed through the same procedure as used for the set (1″).

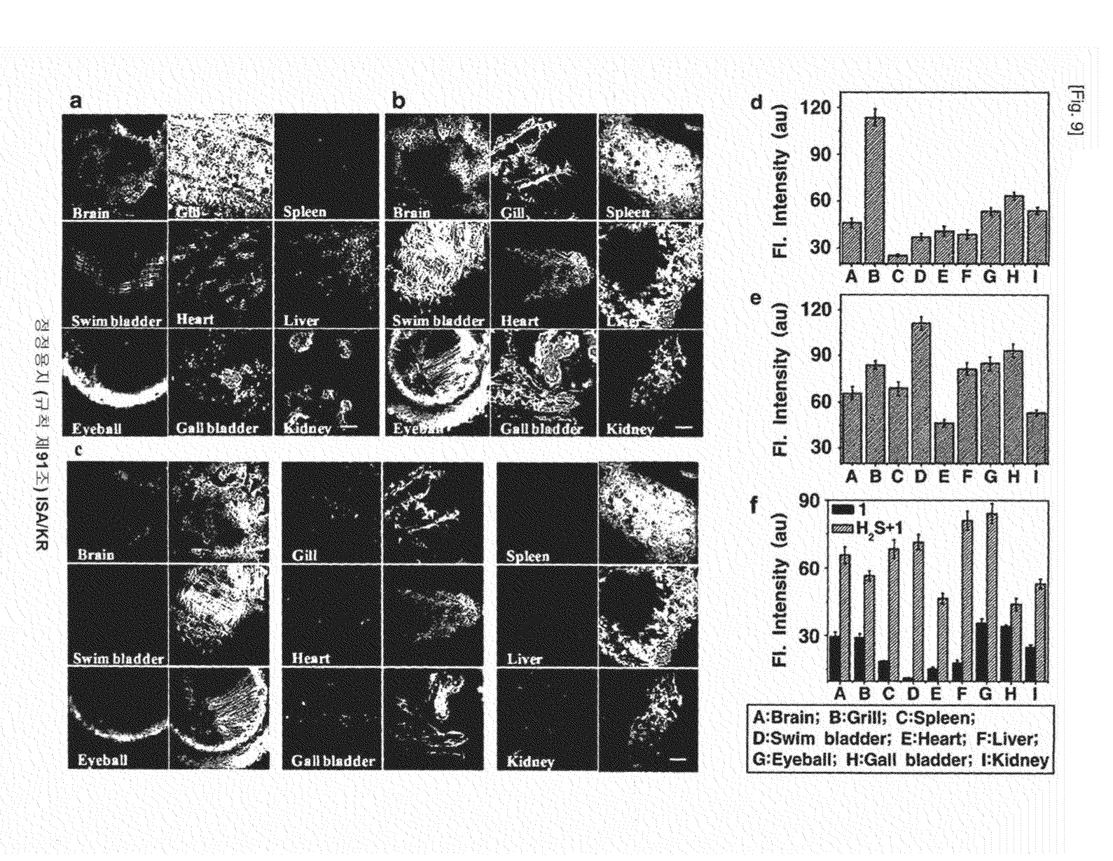

The results of the tissue imaging of the zebrafish are shown in FIG. 9. FIG. 9a shows the result for the set (1″), FIG. 9b shows the result for the set (2″), and FIG. 9c shows the comparison in fluorescence between the sets (1″) and (2″) per organ. From these drawings, the distribution of hydrogen sulfide per organ and a fluorescence change of each organ by external hydrogen sulfide were observed. In FIGS. 9a, 9b and 9c, a scale bar is 50 μm, and FIGS. 9d, 9e, and 9f are obtained by plotting the fluorescence intensities per organ of FIGS. 9a, 9b, and 9c. Here, a vertical axis represents the fluorescence intensity, and a horizontal axis represents each organ.

From the above results, in addition to the distribution of hydrogen sulfide in a living organism, it can also be seen in which organ is the hydrogen sulfide more concentrated under a condition of external treatment of the hydrogen sulfide.

### Example 10

### Confirmation of Cytotoxicity of Compound 2

To confirm the cytotoxicity of Compound 2 according to the present invention, a cytotoxicity experiment in HeLa cells was performed by an MTT method. That is, the Hela cells prepared by the same method as used in Example 7 were treated with Compound 2 at each concentration (0˜100 μM). In addition, to confirm the cytotoxicity, 25 μL of 3-(4,5-dimethldiazol-2-yl)-2, 5-diphenyltetrazolium bromide (MTT) having a concentration of 5 mg/mL was added. The cells were cultured at 37° C. for about 2 hours, treated with 100 μL of a solubilizing solution (50% dimethylformamide, 20% SDS, pH 7.4), and cultured at 37° C. for 24 hours, and then the absorbance was measured at 570 nm.

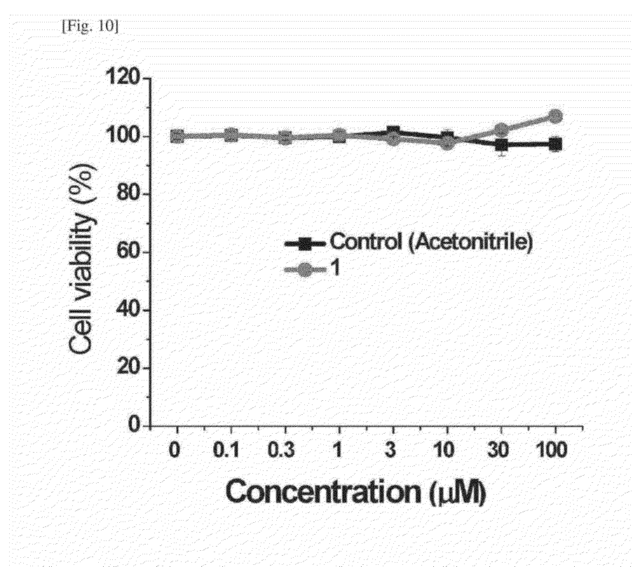

As a result, as shown in FIG. 10, a cell viability was 95% or more until 100 μM, which can be seen to be similar to the control, which was treatment with acetonitrile.

Accordingly, it can be seen that Compound 2 is not toxic to the cells.

### Example 11

### Quantum Chemical Calculation for Selective Reaction Between Compound 2 and Hydrogen Sulfide

Quantum chemical calculation was performed to identify a selective reaction of Compound 2 with hydrogen sulfide. Compound 2 reacted with hydrogen sulfide, resulting in intramolecular cyclization (refer to Example 1 and FIG. 1a). The key point of such cyclization is related with the β-carbon electrophilicity at an enone group of Compound 2 binding to hydrogen sulfide. As the electrophilicity with respect to the β-carbon, which is obtained by calculation, is higher, the quantum chemical calculation value is gradually decreased (decreased to a ‘negative’ value). This means that Compound 2 can easily react with another sulfide, other than hydrogen sulfide. As the electrophilicity with respect to the β-carbon is lower, the quantum chemical calculation value was gradually increased (increased to a ‘positive’ value), which means that Compound 2 can selectively react with the hydrogen sulfide. For convenience of the quantum chemical calculation, a 2-hydroxyethylamino group was removed before calculation, and methoxy groups were introduced to ortho and para positions to confirm the effect of an electron donor group, which was a factor capable of influencing the electrophilicity of the β-carbon. When none of groups are introduced, it is called PF, when two methoxy groups are introduced to ortho positions, it is called P2′, and when methoxy groups are introduced to all of ortho-para positions, it is called P3′. The quantum chemical calculation was performed based on B3LYP-level density functional theory (DFT), and the entire system used a Spartan'08 program package.

According to the calculation, as the calculated value goes to a negative value, the electrophilicity is increased. Therefore, as shown in FIG. 11, it can be seen that the electrophilicity with respect to the β-carbon of the enone is decreased by introducing the methoxy group.

Therefore, when the methoxy groups are introduced to all of the ortho-para positions as shown in Formula 2 of the present invention, it is expected to provide high selectivity, particularly, to hydrogen sulfide.

### Example 12

### Confirmation of Fluorescence Changes Due to Reactions Between Hydrogen Sulfide and Compounds 2, 3 and 4

To prove the results of the quantum chemical calculation, the selectivity of Compounds 2, 3 and 4 to hydrogen sulfide was confirmed under hydrogen sulfide and biological sulfide conditions. That is, the fluorescence changes of Compounds 2, 3 and 4 (10 μM) were observed under the biological sulfide conditions (Na₂S (100 μM, the same as H₂S), glutathione (GSH, 10 mM), cysteine (Cys, 200 μM), homocysteine (Hcy, 50 μM)), a buffer used in the experiment was the same as used in Example 1, an excitation wavelength was 375 nm, and a fluorescence emission wavelength was detected to be 510 nm.

While Compound 2 showed high selectivity to hydrogen sulfide as confirmed in Example 3 and FIG. 3, as shown in FIG. 12a, Compound 3 in which one electron donor group was introduced to an ortho position had relatively lower selectivity to hydrogen sulfide than Compound 2, and as shown in FIG. 12b, Compound 4 in which no electron donor group was introduced did not have selectivity to hydrogen sulfide under the biological sulfide conditions (vertical axis: fluorescence intensity, horizontal axis: time).

Therefore, like the results of the quantum chemical calculation performed in Example 11, it can be seen that the electron donor group has an effect on the selectivity to hydrogen sulfide.

It would be understood by those of ordinary skill in the art that the above descriptions of the present invention are exemplary, and the exemplary embodiments disclosed herein can be easily modified into other specific forms without changing the technical spirit or essential features of the present invention. Therefore, it should be interpreted that the exemplary embodiments described above are exemplary in all aspects, and are not limitative.

### INDUSTRIAL APPLICABILITY

A fluorescent probe of the present invention is a small organic molecule, and can provide a fluorescent signal with high selectivity and sensitivity when binding to hydrogen sulfide. Therefore, the problems of conventionally developed fluorescent probes, such as low substrate selectivity, low sensitivity, and a low response rate, can be overcome, and the distribution of hydrogen sulfide present in the living body can be clearly observed with high resolution using a two-photon fluorescence microscope.

1. A one-photon and/or two-photon fluorescent probe, which is represented by Formula 1: 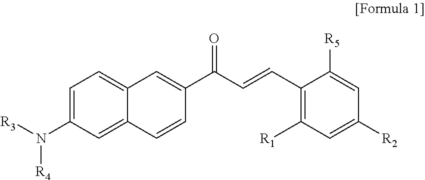
where R₁ is hydrogen, an alkyl, or a substituted C₁₋₃ alkyl, R₂ is hydrogen, an alkyl, or a substituted C₁₋₃ alkyl, R₃ is hydrogen, an alkyl, or a substituted C₁₋₃ alkyl, R₄ is hydrogen or an alkyl, and R₅ is CHO or COCF₃.

2. The probe of claim 1, wherein the probe binds to hydrogen sulfide, thereby exhibiting fluorescence.

3. A method of imaging hydrogen sulfide in cells, comprising the steps of:
(a) injecting the fluorescent probe of claim 1 into cells;
(b) reacting the injected fluorescent probe with the hydrogen sulfide present in the cell, thereby exhibiting fluorescence; and
(c) observing the fluorescence using a one-photon or two-photon fluorescence microscope.

4. A method of manufacturing a one-photon and/or two-photon fluorescent probe for sensing hydrogen sulfide, which is shown in Reaction Formula 1, the method comprising the steps of:
1) preparing a compound of Formula 6 by performing a Heck reaction on a compound of Formula 5 in the presence of a palladium catalyst, and subsequently performing a Bucherer reaction with 2-aminoethanol;
2) preparing a compound of Formula 8 by performing esterification on a compound of Formula 7 in the presence of an acidic catalyst, and subsequently performing bromination and a redox reaction;
3) preparing a compound of Formula 9 by sequentially performing acetal protection and lithium-formylation on the compound of Formula 8;
4) preparing a compound of Formula 10 by performing aldol condensation between the compound of Formula 6 and the compound of Formula 9; and
5) preparing a compound of Formula 2 by a reaction of the compound of Formula 10 under an acidic condition.

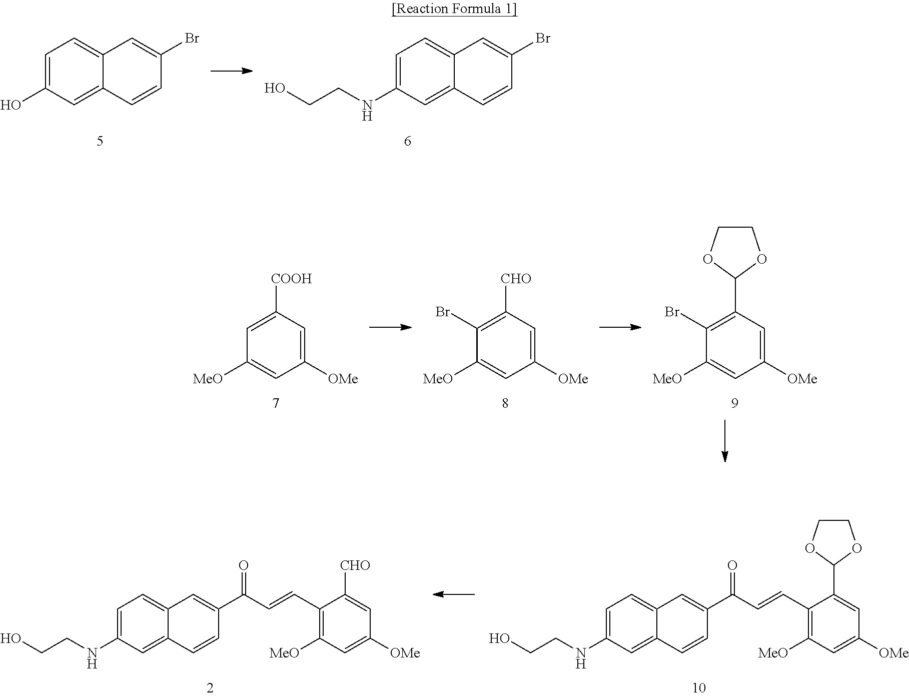

5. A method of manufacturing a one-photon and/or two-photon fluorescent probe for sensing hydrogen sulfide shown by Reaction Formula 2, the method comprising the steps of:
1′) preparing a compound of Formula 12 by performing acetal protection on a compound of Formula 11;
2′) preparing a compound of Formula 13 by lithium-formylation of the compound of Formula 12;
3′) preparing a compound of Formula 14 by aldol condensation between the compound of Formula 13 and the compound of Formula 6 prepared in step 1) of claims 4; and
4′) preparing a compound of Formula 3 by performing a reaction of the compound of Formula 14 under an acidic condition.

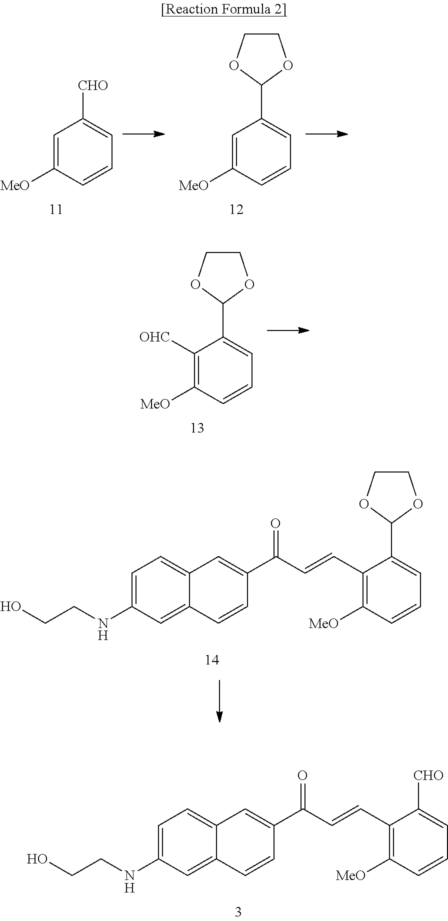 [SMILES: COc1cccc(C=O)c1 ; COc1cccc(C2OCCO2)c1 ; COc1cccc(C2OCCO2)c1C=O ; COc1cccc(C2OCCO2)c1/C=C/C(=O)c1ccc2cc(NCCO)ccc2c1 ; COc1cccc(C=O)c1/C=C/C(=O)c1ccc2cc(NCCO)ccc2c1]

6. A method of manufacturing a one-photon and/or two-photon fluorescent probe for sensing hydrogen sulfide shown by Reaction Formula 3, the method comprising the steps of:
1″) preparing a compound of Formula 16 by sequentially performing acetal protection and lithium-formylation on a compound of Formula 15;
2″) preparing a compound of Formula 17 by aldol condensation between the compound of Formula 16 and the compound of Formula 6 prepared in step 1) of claims 4; and
3″) preparing a compound of Formula 4 by a reaction of the compound of Formula 17 under an acidic condition. 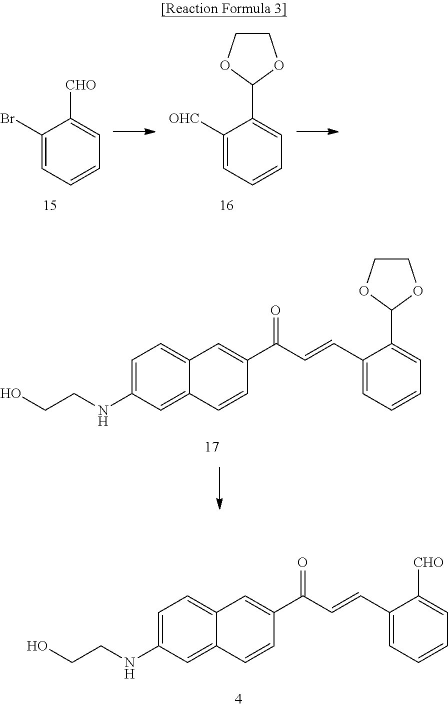 [SMILES: O=Cc1ccccc1Br ; O=Cc1ccccc1C1OCCO1 ; O=C(/C=C/c1ccccc1C1OCCO1)c1ccc2cc(NCCO)ccc2c1 ; O=Cc1ccccc1/C=C/C(=O)c1ccc2cc(NCCO)ccc2c1]
